(19) United States
(12) Patent Application Publication
(10) Pub. No.: US 2024/0374434 A1
(43) Pub. Date: Nov. 14, 2024

(54) NEGATIVE PRESSURE DRESSING WITH DIFFERENTIAL COLLAPSING FORCE FOR WOUND CLOSURE

(71) Applicant: KCI Manufacturing Unlimited Company, Athlone, Co., Westmeath (IE)

(72) Inventors: Christopher J. CARROLL, San Antonio, TX (US); Jonathan G. REHBEIN, San Antonio, TX (US); Michael R. OLIVA, San Antonio, TX (US); Javier GONZALEZ, San Antonio, TX (US)

(21) Appl. No.: 18/688,718

(22) PCT Filed: Aug. 23, 2022
(86) PCT No.: PCT/IB2022/057870

§ 371 Date: Mar. 1, 2024

Related U.S. Application Data
(60) Provisional application No. 63/241,160, filed on Sep. 7, 2021.

(51) Int. Cl. A61F 13/0203

(52) U.S. Cl. CPC A61F 13/0203

(57) ABSTRACT

A negative pressure dressing includes a first foam section, a second foam section, and a longitudinal seam joining the first foam section to the second foam section. The longitudinal seam has a higher rigidity than the first foam section and the second foam section.

### CROSS-REFERENCE TO RELATED APPLICATIONS

This application is a U.S. National Stage Entry of International Application No. PCT/IB2022/057870, filed on Aug. 23, 2022, which claims the benefit of priority to U.S. Provisional Application No. 63/241,160, filed on Sep. 7, 2021, which is incorporated herein by reference in its entirety.

### BACKGROUND

The present disclosure relates generally to negative pressure therapy, for example negative pressure therapy for an open abdominal wound. In negative pressure therapy (NPT), a wound is subjected to a negative pressure (i.e., lower than atmospheric pressure), which can promote wound healing. In some case, negative pressure therapy systems are adapted for treatment of open wounds, i.e., wounds in which tissue other than skin is exposed to the ambient environment (and to the wound dressing). In such cases, it may desirable to promote closure of the open wound.

### SUMMARY

One implementation of the present disclosure is a negative pressure dressing. The dressing includes a first foam section, a second foam section, and a longitudinal seam joining the first foam section to the second foam section, wherein the longitudinal seam has a higher rigidity than the first foam section and the second foam section.

In some embodiments, the longitudinal seam is configured to cause the first foam section and the second foam section to collapse more in a medial direction of the dressing perpendicular to the longitudinal seam than in a longitudinal direction parallel with the longitudinal seam when subjected to a negative pressure. The longitudinal seam may cause the first foam section and the second foam section to exert, when subjected to a negative pressure, a medial force in a direction perpendicular to the longitudinal seam which exceeds a longitudinal force exerted by the first foam section and the second foam section in a direction parallel with the longitudinal seam.

In some embodiments, the longitudinal seam is porous. The longitudinal seam may include glue and/or a panel of non-foam material. In some embodiments, the negative pressure dressing also includes a third foam section and an additional longitudinal seam. The third foam section is separated from the second foam section by the additional longitudinal seam. The additional longitudinal seam has a higher rigidity than the third foam section and the second foam section. In some embodiments, the longitudinal seam and the additional longitudinal seam are positioned symmetrically across an imaginary longitudinal center line of the second foam section.

In some embodiments, the dressing also includes a fenestrated visceral protective layer configured to be placed over an open abdominal wound to separate the first foam section and the second foam section from the open abdominal wound. The dressing may also include a drape sealable over a wound to define an airtight volume containing the first foam section, the second foam section, and the longitudinal seam.

Another implementation of the present disclosure is a negative pressure dressing that includes a first foam section having a first characteristic, a second foam section having the first characteristic, and a third foam section having a second characteristic different than the first characteristic. The third section separates the first section from the second section along a longitudinal direction of the dressing. When the dressing is subjected to a negative pressure, a non-zero difference between the second characteristic and the first characteristic causes the dressing to collapse to a greater extent in a medial direction perpendicular to the longitudinal direction as compared to an embodiment with zero difference between the second characteristic and the first characteristic.

In some embodiments, the first characteristic is a first thickness and the second characteristic is a second thickness, with the second thickness being greater than the first thickness. In some embodiments, the first characteristic is a first density and the second characteristic is a second density, with the second density being greater than the first density. The greater extent may be a greater force or a greater amount (e.g., distance) of compression. The non-zero difference between the second characteristic and the first characteristic may cause the dressing to collapse to a lesser extent in the longitudinal direction as compared to an embodiment with zero difference between the second characteristic and the first characteristic.

Another implementation of the present disclosure is a negative pressure therapy system that includes a negative pressure source and a dressing. The dressing is fluidly communicable with the negative pressure source. The dressing includes a wound contact layer configured to contact an open wound of a patient, a manifold layer, and a drape layer coupled to the wound contact layer such that the manifold layer is between the wound contact layer and the drape layer. The manifold layer includes a first foam section, a second foam section, and a longitudinal seam joining the first foam section to the second foam section. The longitudinal seam has a higher rigidity than the first foam section and the second foam section.

In some embodiments, the negative pressure source is operable to establish a negative pressure in the manifold layer. The longitudinal seam is configured to cause the first foam section and the second foam section to collapse more in a medial direction of the dressing perpendicular to the longitudinal seam than in a longitudinal direction parallel with the longitudinal seam when the negative pressure is established in the manifold layer. In some embodiments, the longitudinal seam is configured to allow communication of negative pressure across the longitudinal seam from the first foam section to the second foam section. In some embodiments, the longitudinal seam comprises a panel of a non-foam material.

This summary is illustrative only and is not intended to be in any way limiting.

### BRIEF DESCRIPTION OF THE FIGURES

The disclosure will become more fully understood from the following detailed description. taken in conjunction with the accompanying figures, wherein like reference numerals refer to like elements, in which:

FIG. 1 is a perspective view of a negative pressure therapy (NPT) system shown as a kit, according to some embodiments.

FIG. 2 is a schematic illustration of the NPT system of FIG. 1 in use treating an open wound, according to some embodiments.

FIG. 3 is an illustration of a manifolding layer of the negative pressure therapy system of FIG. 1, according to some embodiments.

FIG. 4 is a cross-section schematic view of the manifolding layer of the negative pressure therapy system of FIG. 1, according to various embodiments.

FIG. 5 is a cross-section schematic view of an alternative embodiment of a manifolding layer for the negative pressure therapy system of FIG. 1, according to some embodiments.

FIG. 6 is a cross-section schematic view of another alternative embodiment of a manifolding layer for the negative pressure therapy system of FIG. 1, according to some embodiments.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the figures, a negative pressure therapy (NPT) system with a dressing configured to promote wound closure is shown, according to various embodiments. The dressing is configured to be applied of over an open wound, for example an abdominal wound and enables a negative pressure (relative to atmospheric pressure) to be communicated the open wound.

In the examples herein, the dressing includes a manifold layer configured to collapse primarily in a medial direction (e.g., toward a center line along a longitudinal axis of the manifold layer), thereby exerting a medial force on tissue which can promote wound closure. The manifold layer includes longitudinal seams, panels, regions, etc. in various embodiments that increase the medial collapse of the manifold layer under negative pressure and a medial force created by such collapse, for example relative to embodiments without such longitudinal seams, panels, regions, etc. and/or relative to collapse and forces in other directions. This increased medial force has beneficial therapeutic effects, for example by drawing together regions of skin on either side of a wound. These and other features are described in detail below.

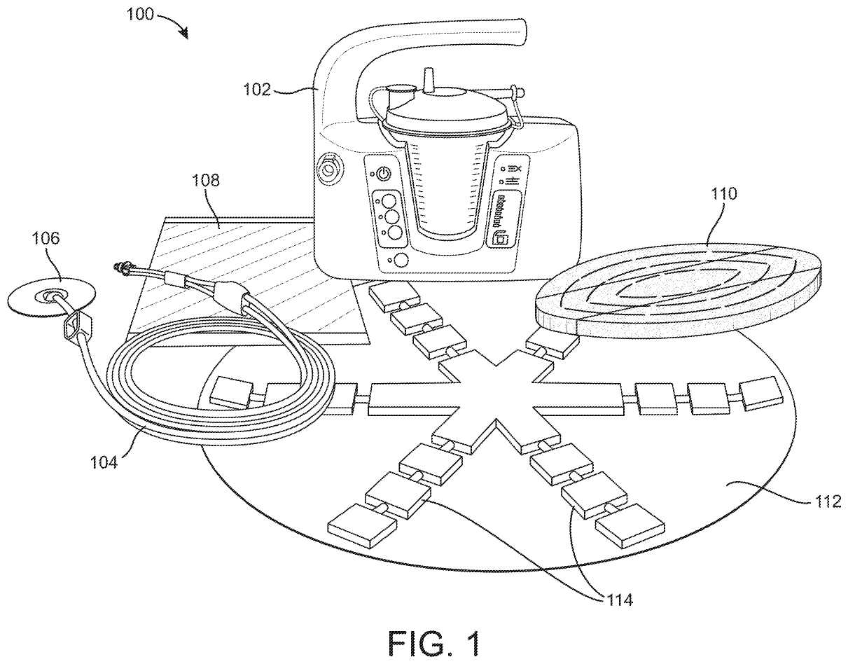

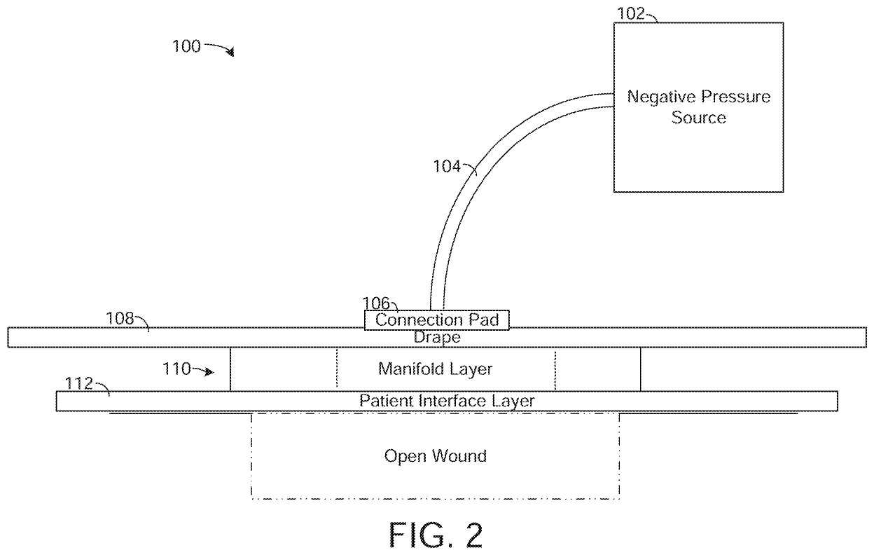

Referring now to FIGS. 1-2, depictions of an NPT system 100 are shown, according to some embodiments. FIG. 1 shows an isometric view of the NPT system 100 as a kit, for example before application to a patient's wound to treat the wound. FIG. 2 shows the NPT system 100 in a schematic side view and in use treating a wound. The NPT system 100 includes a negative pressure source (e.g., pump, therapy unit) 102, tubing 104 with a connection pad 106, a drape 108, a manifold layer 110, and a patient interface layer (wound contact layer) 112. The NPT system 100 is configured to provide negative pressure therapy to a wound, including an open abdominal wound, while exerting a medial closing force on the wound. The drape 108, manifold layer 110, and the patient interface layer 112 can be considered to be components of a dressing (e.g., a negative pressure dressing).

As shown in FIG. 1, the negative pressure source (e.g., pump, therapy unit) 102, the tubing 104 with the connection pad 106, the drape 108, the manifold layer 110, and the patient interface layer 112 can be provided as separate elements (e.g., during shipping, storage, delivery, etc.) in a kit. The components of the NPT system 100 can then be applied to a patient as shown in FIG. 2. As shown in FIG. 2, the patient interface layer 112 is in contact with a patient wound, for example positioned over an open wound (e.g., spanning an entirety of the open wound). The manifold layer 110 is adjacent the patient interface layer 112 and the drape 108 is adjacent the manifold layer 110, such that the manifold layer 110 is between the drape 108 and the patient interface layer 112. The drape 108 and/or the patient interface layer 112 may include adhesive configured to couple the drape 108 to the patient interface layer 112 and/or the couple the drape 108 and/or the patient interface layer 112 to the patient. The drape 108 is sealable over the wound to define a substantially airtight volume containing the manifold layer 110.

The connection pad 106 is configured to couple to the drape 108, for example over a hole formed in the drape 108 to place the tubing 104 in fluid communication with the manifold layer 110. The tubing 104 is configured to be connected to the negative pressure source 102, for example as shown in FIG. 2. FIG. 2 illustrates the negative pressure source 102 in fluid communication with the manifold layer 110 via the tubing 104 and the connection pad 106. The negative pressure source 102 can operate to generate a negative pressure in the manifold layer 110, for example by pumping, pulling, drawing, etc. air out of the manifold layer 110 via the tubing 104 so that the air pressure in the manifold layer 110 is reduced below atmospheric pressure. The negative pressure source 102 can also draw wound exudate, debris, fluids, etc. out of the wound via the tubing 104 and collect such exudate, debris, fluid, etc. in a canister of the negative pressure source 102. In some embodiments, the negative pressure source 102 is also operable to provide instillation fluid to the wound via the tubing 104.

The patient interface layer 112 is configured to contact the wound, for example substantially without sticking to the wound while preventing growth of tissue into the patient interface layer 122. The patient interface layer 112 can be a fenestrated film configured to allow fluid and airflow across the patient interface layer 112 (e.g., through a plurality of apertures, perforations, slits, windows, etc.). A negative pressure at the manifold layer 110 can thus be communicated to the wound across the patient interface layer 112. As shown in FIG. 1, the patient interface layer 112 can include manifolding pathways 114 arranged as arms or spokes extending from a central region of the patient interface layer 112. The manifolding pathways 114 distribute negative pressure (or other air or fluid flow) to extremities of the patient interface layer 112, thereby facilitating exposure of a large extent of the wound to negative pressure. The manifolding pathways 114 may be made of an open-celled foam and are communicable with the manifold layer 110 when assembled for use as shown in FIG. 2.

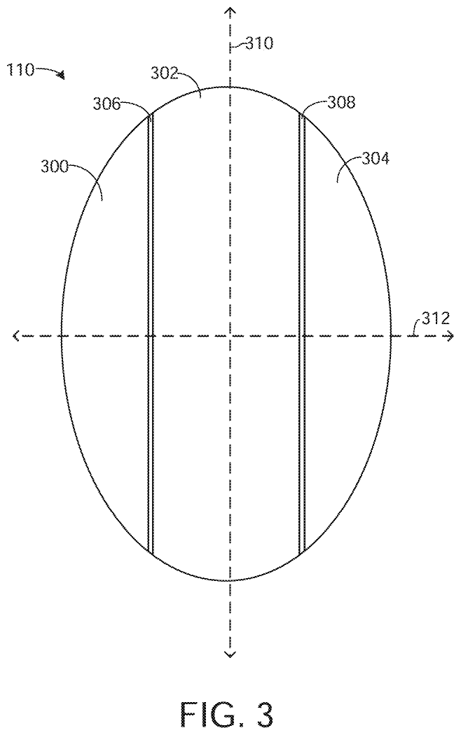

As shown in FIG. 1, the manifold layer 110 has an elliptical or oblong shape having a length in a longitudinal direction greater than a width in a lateral direction. In other embodiments, the manifold layer 110 has another shape (e.g., rectangular, trapezoidal, pentagonal, hexagonal, etc.). The manifold layer 110 provides for the communication of air and pressure therethrough, such that the manifold layer 110 internally regulates to a substantially uniform pressure across a full extent of the manifold layer 110. For example, the manifold layer 110 may include an open celled foam which allows air and fluid flow therethrough, including under high negative pressures (e.g., at a negative pressure of −125 mmHg, −150 mmHg, etc.). As described in further detail below with reference to FIGS. 3-6, the manifold layer 110 is configured to collapse under negative pressure to different extents in different directions, thereby exerting a desired force profile on patient tissue. To provide such behavior, and as described below, the manifold layer 110 is shown as being made up of multiple foam sections joined together by longitudinal seams. When made of one or more foam portions, the manifold layer 110 may be referred to as a foam layer.

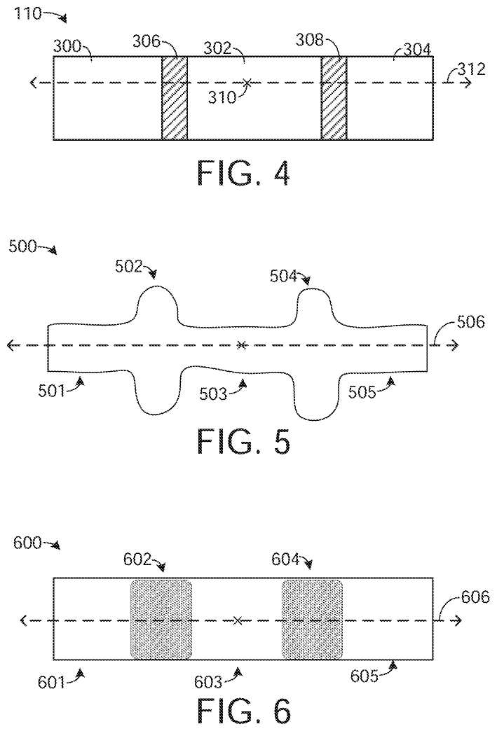

Referring now to FIGS. 3-4, a top view (FIG. 3) and a cross-sectional side view (FIG. 4) of the manifold layer 110 is shown, according to some embodiments. The manifold layer 110 is shown as including a first foam section 300, a second foam section 302, a third foam section 304, a first longitudinal seam 306 joining the first foam section 300 to the second foam section 302, and a second longitudinal seam 308 joining the second foam section 302 to the third foam section 304. The second foam section 302 is between the first longitudinal scam 306 and the second longitudinal scam 308. The second foam section 302 is also between the first foam section 300 and the third foam section 304. As shown in FIGS. 3-4, the second foam section 302 is a central region or portion of the manifold layer 110, while the first foam section 300 and the third foam section 304 are wings, side portions, side regions, etc. of the manifold layer 110. In the example of FIG. 3, the first longitudinal scam 306 and the second longitudinal seam 308 are symmetric across an imaginary center line (longitudinal axis) 310 of the manifold layer 110, as are the first foam section 300 and the third foam section 304. The first longitudinal seam 306 and the second longitudinal seam 308 extend across a thickness of the manifold layer 110 (e.g., from a patient-facing side to a non-patient facing side).

The first foam section 300, the second foam section 302, and the third foam section 304 may be made of a foam material having first characteristics (e.g., density, thickness, compressibility, rigidity, etc.) that are substantially the same for the three foam sections 300-304. The foam material may be an open-celled foam, for example GRANUFOAM® marketed by KCl. The foam material allows fluid to flow therethrough and allows negative pressure to be communicated therethrough. The foam material partially collapses under negative pressure while retaining its manifolding ability even under high negative pressures (e.g., beyond-150 mmHg).

The first longitudinal seam 306 and the second longitudinal seam 308 are made of a material having one or more different characteristics than the foam sections 300-304 (e.g., a non-foam material). In some embodiments, the first longitudinal seam 306 and the second longitudinal seam 308 have a higher rigidity and lower compressibility than the foam sections 300-304. The first longitudinal seam 306 and the second longitudinal seam 308 can provide an “accordion-like” collapsing behavior for the manifold layer 110 as further described below.

For example, in some embodiments the first longitudinal seam 306 and the second longitudinal seam 308 are formed of an adhesive (e.g., glue) which adheres the first foam section 300 to the second foam section 302 and adheres the second foam section 302 to the third foam section 304. The adhesive may form panels of adhesive (glue) with higher rigidity than the foam sections 300-304. In some embodiments, the first longitudinal seam 306 is a first panel of glue and the second longitudinal seam 308 is a second panel of glue. The panels of glue are preferably porous such that fluid and pressure can be communicated across the first longitudinal seam 306 and the second longitudinal seam 308.

In other embodiments, the first longitudinal seam 306 and the second longitudinal seam 308 are formed by heat—or flame-welding the first foam section 300 to the second foam section 302 and the second foam section 302 to the third foam section 304. The heat—or flame-welding can form panels or struts of higher-density, partially-melted foam at the first longitudinal seam 306 and the second longitudinal seam 308 with a higher rigidity than the first foam section 300, the second foam section 302, and the third foam section 304

The first longitudinal seam 306 and the second longitudinal seam 308 can include one or alternative or additional materials in various embodiments. For example, a metal (e.g., steel, copper) or plastic may be used to form plates or panels which are included in the first longitudinal seam 306 and the second longitudinal seam 308 in some embodiments. Such plates or panels can include holes, windows, perforations, etc. which allow air and fluid flow therethrough.

When the manifold layer is under negative pressure, the first longitudinal seam 306 and the second longitudinal seam 308 restrict collapse of the manifold layer in certain directions, thereby promoting collapse in another direction. In particular, their rigidity and/or lower compressibility of the first longitudinal seam 306 and the second longitudinal seam 308 relative to the foam sections 300-304 cause the first longitudinal seam 306 and the second longitudinal seam 308 to collapse less than the foam sections 300-304. Because the first longitudinal seam 306 and the second longitudinal seam 308 are oriented in a longitudinal direction (parallel with longitudinal axis 310), the resistance to collapse of the first longitudinal seam 306 and the second longitudinal seam 308 reduce collapse of the manifold layer 110 in the longitudinal direction. Additionally, because the first longitudinal seam 306 and the second longitudinal seam 308 extend across a thickness of the manifold layer 110, the first longitudinal seam 306 and the second longitudinal seam 308 resist reduction in thickness of the manifold layer 110 (i.e., into the page from the perspective of FIG. 3). However, the first longitudinal seam 306 and the second longitudinal seam 308 do not extend in a lateral direction of the dressing and therefore do not resist collapse perpendicular to the first longitudinal seam 306 and the second longitudinal seam 308 (i.e., collapse parallel with a lateral axis 312). Because medial collapse—i.e., towards a center line (longitudinal axis) 310 in a lateral direction (along lateral axis 312) perpendicular to the longitudinal axis 310—is not resisted by the first longitudinal seam 306 and the second longitudinal seam 308, medial collapse is caused to exceed other directions of collapse of the manifold layer 110.

Such increased collapse may characterized by increased medial force exerted on surrounding structures (e.g., on patient anatomy) by the manifold layer 110 and/or increased distance of compression (e.g., an increased change in size) in the medial direction as compared to embodiments in which the first longitudinal seam 306 and the second longitudinal seam 308 are omitted. Additionally or alternatively, such increased collapse may characterized by increased medial force (perpendicular to the longitudinal seams 306-308) exerted on surrounding structures (e.g., on patient anatomy) by the manifold layer 110 and/or increased distance of compression (e.g., an increased change in size) in the medial direction (i.e., perpendicular to the longitudinal seams 306-308) as compared to the force and/or distance of collapse in the longitudinal direction.

The characteristics (e.g., rigidity) of the first longitudinal seam 306 and the second longitudinal seam 308 thereby cause increased medial collapse when the manifold layer 110 is exposed to negative pressure during use of the NPT system 100, which may desirably increase a closure force on a wound, e.g., a force pulling healthy skin together over an open portion of a wound, thereby facilitating healing of the wound and wound closure. For example, such a dressing may be advantageous in scenarios where a suturing approach to closing a wound is not clinically appropriate.

Referring now to FIG. 5, a schematic side view of a manifold layer 500 is shown, according to some embodiments. The manifold layer 500 can be used in place of the manifold layer 110 of FIGS. 1-4 in various embodiments, and is configured to achieve the same or similar advantages (e.g., differential collapse providing increased medial closure forces).

As shown in FIG. 5, the manifold layer 500 includes a first foam section 501, a second foam section 502, a third foam section 503, a fourth foam section 504, and a fifth foam section 505. The foam sections 501-505 are arranged sequentially, with the second foam section 502 separating the first foam section 501 from the third foam section 503 and the fourth foam section 504 separating the third foam section 503 from the fifth foam section 505. The second foam section 502 and the fourth foam section 504 can extend along a longitudinal direction, for example in a similar manner as described above for the longitudinal seams 306, 308. In the example shown, the foam sections 501-505 are formed from a single, continuous mass of foam (e.g., cut from one block, molded together so as to be formed integrally, etc.).

As shown in FIG. 5, the second foam section 502 and the fourth foam section 504 have a different characteristic than the first foam section 501, the third foam section 503, and the fifth foam section 505. In particular, in the example of FIG. 5, the second foam section 502 and the fourth foam section 504 are shown has having a greater thickness than the first foam section 501, the third foam section 503, and the fifth foam section 505. That is, the manifold layer 500 is narrower at the first foam section 501, the third foam section 503, and the fifth foam section 505 as compared to the second foam section 502 and the fourth foam section 504.

The difference in thickness between the second foam section 502 and the fourth foam section 504 and the first foam section 501, the third foam section 503, and the fifth foam section 505 provides increased resistance to collapse in the longitudinal direction of the manifold layer 500 (into the page as shown in FIG. 5) and in a direction of the thickness of the manifold layer 500 (vertically from the perspective of FIG. 5), thereby encouraging, enhancing, increasing, etc. medial collapse of the manifold layer 500 in a lateral direction (i.e., toward a center of the dressing along axis 506 shown in FIG. 5). The extent of medial collapse is increased by the variation in thickness across the manifold layer 500, for example relative to an embodiment with a uniform thickness and/or relative to collapse in other directions.

Referring now to FIG. 6, a schematic side view of a manifold layer 600 is shown, according to some embodiments. The manifold layer 600 can be used in place of the manifold layer 110 of FIGS. 1-4 in various embodiments, and is configured to achieve the same or similar advantages (e.g., differential collapse providing increased medial closure forces).

As shown in FIG. 6, the manifold layer 600 includes a first foam section 601, a second foam section 602, a third foam section 603, a fourth foam section 604, and a fifth foam section 605. The foam sections 601-605 are arranged sequentially, with the second foam section 602 separating the first foam section 601 from the third foam section 603 and the fourth foam section 604 separating the third foam section 603 from the fifth foam section 605. The second foam section 602 and the fourth foam section 604 can extend along a longitudinal direction, for example in a similar manner as described above for the longitudinal seams 306, 308. In the example shown, the foam sections 601-605 are formed from a single, continuous mass of foam (e.g., cut from one block, molded together so as to be formed integrally, etc.).

As shown in FIG. 6, the second foam section 602 and the fourth foam section 604 have a different characteristic than the first foam section 601, the third foam section 603, and the fifth foam section 605. In particular, in the example of FIG. 6, the second foam section 602 and the fourth foam section 604 have a higher density than the first foam section 601, the third foam section 603, and the fifth foam section 605. For example, the manifold layer 600 can be formed, for example, via felting process in which the second foam section 602 and the fourth foam section 604 are compressed and heated such that the second foam section 602 and the fourth foam section 604 modified to hold their compressed shape and density. For example, during production, the manifold layer 600 may first be shaped similar to the manifold layer 500 of FIG. 5, and then the second foam section 602 and the fourth foam section 604 are felted to achieve a greater density and similar thickness as the first foam section 601, the third foam section 603, and the fourth foam section 604. The manifold layer 500 thus has alternating sections of a first density and a second density, for example where the second density is in a range approximately between 1.5 and 3 times the first density.

The difference in density between the second foam section 602 and the fourth foam section 604 and the first foam section 601, the third foam section 603, and the fifth foam section 605 provides increased resistance to collapse in the longitudinal direction of the manifold layer 600 (into the page as shown in FIG. 6) and in a direction of the thickness of the manifold layer 600 (vertically from the perspective of FIG. 6), thereby encouraging, enhancing, increasing, etc. medial collapse of the manifold layer 600 in a lateral direction (i.e., toward a center of the dressing along axis 606 shown in FIG. 6). The extent of medial collapse is increased by the variation in density across the manifold layer 600, for example relative to an embodiment with a uniform thickness and/or relative to collapse in other directions.

In other embodiments, other treatments of the second foam section 602 and the fourth foam section 604 can provide similar effects by providing different characteristics across the manifold layer 600. For example, the second foam section 602 and the fourth foam section 604 may be chemically treated or impregnated with another material to have a higher rigidity than the first foam section, the third foam section 603, and the fifth foam section 604, for example. As yet another embodiment, the manifold layer 600 may be selectively weakened in certain directions, for example provided with fenestrations, perforations, open spaces, cut-outs, windows, etc. that are arranged to encourage collapse of the manifold layer in one direction (e.g., via closure of the fenestrations, perforations, etc.) to a greater extent than in other directions. Many such variations are with the scope of the present disclosure.

The examples herein include an example number of foam sections (e.g., three sections with two seams as in FIGS. 3-4, five sections as in FIGS. 5-6). Various other numbers of foam sections, seams, sections of different characteristics, etc. may be provided in various embodiments. For example, in some embodiments the manifold layer 110 includes two foam sections and one longitudinal seam. In other embodiments, the manifold layer 110 may include four foam sections with three longitudinal seams, five foam sections with four longitudinal seams, sight foam sections with five longitudinal seams, etc. Additionally, different orders of the components of the manifold layers 110, 500, 600 may be possible. For example, in some embodiments, the manifold layer 500 may be modified to have three thicker sections separated by two narrower sections. Many such variations are within the scope of the present disclosure.

As utilized herein with respect to numerical ranges, the terms “approximately,” “about.” “substantially,” and similar terms are intended to have a broad meaning in harmony with the common and accepted usage by those of ordinary skill in the art to which the subject matter of this disclosure pertains. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process. Accordingly, these terms should be interpreted as indicating that insubstantial or inconsequential modifications or alterations of the subject matter described and claimed are considered to be within the scope of the disclosure as recited in the appended claims.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

1. A negative pressure dressing comprising:
a first foam section;
a second foam section; and
a longitudinal seam joining the first foam section to the second foam section, wherein the longitudinal seam has a higher rigidity than the first foam section and the second foam section.

2. The negative pressure dressing of claim 1, wherein the longitudinal seam is configured to cause the first foam section and the second foam section to collapse more in a medial direction of the dressing perpendicular to the longitudinal seam than in a longitudinal direction parallel with the longitudinal seam when subjected to a negative pressure.

3. The negative pressure dressing of claim 1, wherein the longitudinal seam is configured to cause the first foam section and the second foam section to exert, when subjected to a negative pressure, a medial force in a direction perpendicular to the longitudinal seam which exceeds a longitudinal force exerted by the first foam section and the second foam section in a direction parallel with the longitudinal seam.

4. The negative pressure dressing of claim 1, wherein the longitudinal seam is porous.

5. The negative pressure dressing of claim 1, wherein the longitudinal seam comprises glue.

6. The negative pressure dressing of claim 1, wherein the longitudinal seam comprises a panel of a non-foam material.

7. The negative pressure dressing of claim 1, further comprising:
a third foam section; and
an additional longitudinal seam, wherein the third foam section is separated from the second foam section by the additional longitudinal seam, the additional longitudinal seam having a higher rigidity than the third foam section and the second foam section.

8. The negative pressure dressing of claim 7, wherein the longitudinal seam and the additional longitudinal seam are positioned symmetrically across an imaginary longitudinal center line of the second foam section.

9. The negative pressure dressing of claim 1, further comprising a fenestrated visceral protective layer configured to be placed over an open abdominal wound to separate the first foam section and the second foam section from the open abdominal wound.

10. The negative pressure dressing of claim 1, further comprising a drape sealable over a wound to define an airtight volume containing the first foam section, the second foam section, and the longitudinal seam.

11. A negative pressure dressing, comprising:
a first foam section having a first characteristic;
a second foam section having the first characteristic; and
a third foam section having a second characteristic different than the first characteristic, the third foam section separating the first foam section from the second foam section along a longitudinal direction of the dressing;
wherein, when the dressing is subjected to a negative pressure, a non-zero difference between the second characteristic and the first characteristic causes the dressing to collapse in a medial direction perpendicular to the longitudinal direction to a greater extent than a zero difference between the second characteristic and the first characteristic.

12. The negative pressure dressing of claim 11, wherein the first characteristic is a first thickness and the second characteristic is a second thickness, the second thickness greater than the first thickness.

13. The negative pressure dressing of claim 11, wherein the first characteristic is a first density and the second characteristic is a second density, the second density greater than the first density.

14. The negative pressure dressing of claim 11, wherein the collapse of the dressing to the greater extent includes a greater force.

15. The negative pressure dressing of claim 11, wherein the collapse of the dressing to the greater extent includes a greater distance of compression.

16. The negative pressure dressing of claim 11, wherein the non-zero difference between the second characteristic and the first characteristic causes the dressing to collapse to a lesser extent in the longitudinal direction compared to the zero difference between the second characteristic and the first characteristic.

17. A negative pressure therapy system, comprising:
a negative pressure source;
a dressing fluidly communicable with the negative pressure source, the dressing comprising:
a wound contact layer configured to contact an open wound of a patient,
a manifold layer, and
a drape layer coupled to the wound contact layer such that the manifold layer is between the wound contact layer and the drape layer;
wherein the manifold layer comprises:
a first foam section,
a second foam section, and
a longitudinal seam joining the first foam section to the second foam section, wherein the longitudinal seam has a higher rigidity than the first foam section and the second foam section.

18. The negative pressure therapy system of claim 17, wherein:
the negative pressure source is operable to establish a negative pressure in the manifold layer; and
the longitudinal seam is configured to cause the first foam section and the second foam section to collapse more in a medial direction of the dressing perpendicular to the longitudinal seam than in a longitudinal direction parallel with the longitudinal seam when the negative pressure is established in the manifold layer.

19. The negative pressure therapy system of claim 17, wherein the longitudinal seam is configured to allow communication of negative pressure across the longitudinal seam from the first foam section to the second foam section.

20. The negative pressure therapy system of claim 18, wherein the longitudinal seam comprises a panel of a non-foam material.
